(19) United States
(12) Patent Application Publication
(10) Pub. No.: US 2008/0189291 A1
(43) Pub. Date: Aug. 7, 2008

(54) System for measuring and displaying vital signs and method therefor

(76) Inventor: Kent T.J. Hsu, Taipei (TW)

Correspondence Address: TROXELL LAW OFFICE PLLC, SUITE 1404, 5205 LEESBURG PIKE, FALLS CHURCH, VA, 22041, US

(21) Appl. No.: 11/700,960

(22) Filed: Feb. 1, 2007

(51) Int. Cl. G06F 7/00; A61B 5/00

(52) U.S. Cl. 707/10; 600/301

(57) ABSTRACT

A system and method for measuring and displaying vital signs are provided. A band is wrapped around the wrist of a person. The band includes a first measurement device for measuring blood pressure of the person. A device electrically connected to the band includes a second measurement device for measuring body temperature, pulse, and total calories of the person by contacting. Data about the measurements is sent to a user interface embedded in the device prior to sending to a display associated with the device for showing. The user interface is adapted to process data about the measurements in a spreadsheet, process data about the measurements into files to be stored in a database of a web server, and link to a health care provider via the database. The device is implemented as a computer and a mouse, a video game console and a wireless controller, or a mobile phone.

### BACKGROUND OF THE INVENTION

1. Field of Invention

The invention relates to the measurement of vital signs and more particularly to a system for measuring and displaying vital signs including body temperature, pulse, blood pressure, and total calories and a method therefor.

2. Description of Related Art

It is typical for a person to go to a health care provider (e.g., hospital) to take a physical examination. However, taking a physical examination is a time consuming thing as viewed by some people. Further, some people may desire to know only a few things about his or her health rather than going to a hospital to take a complete physical examination. Thus, it is desirable to provide a simple system which takes advantage of one or more conventional devices for measuring and displaying vital signs and a method associated therewith.

### SUMMARY OF THE INVENTION

It is therefore one object of the invention to provide a system for measuring and displaying vital signs including body temperature, pulse, blood pressure, and total calories of a person wherein the system takes advantage of one or more conventional devices (e.g., a computer and a mouse and wrist band assembly, a video game console (e.g., “Wii”) and a wireless controller (e.g., “Wii” remote), or a mobile phone) being used or played by the person.

It is another object of the invention to provide a method for measuring and displaying vital signs including body temperature, pulse, blood pressure, and total calories of a person wherein the method is performed on a system which takes advantage of one or more conventional devices (e.g., a computer and a mouse and wrist band assembly, a video game console (e.g., “Wii”) and a wireless controller (e.g., “Wii” remote), or a mobile phone) being used or played by the person.

The above and other objects, features and advantages of the invention will become apparent from the following detailed description taken with the accompanying drawings.

### BRIEF DESCRIPTION OF THE DRAWINGS

FIG. 1 is a perspective view of a first preferred embodiment of the invention incorporating a mouse and wrist band assembly and a personal computer;

FIG. 2 is a block diagram of the first preferred embodiment of the invention;

FIG. 3 is a perspective view of a second preferred embodiment of the invention incorporating a video game console (e.g., “Wii”) and a remote controller (e.g., “Wii” remote);

FIG. 4 is a block diagram of the second preferred embodiment of the invention;

FIG. 5 is a perspective view of a third preferred embodiment of the invention incorporating a mobile phone;

FIG. 6 is a block diagram of the third preferred embodiment of the invention;

FIG. 7 is a flowchart depicting a process for measuring and displaying vital signs according to the first preferred embodiment of the invention;

FIG. 8 is a flowchart depicting a process for measuring and displaying vital signs according to the second preferred embodiment of the invention; and

FIG. 9 is a flowchart depicting a process for measuring and displaying vital signs according to the third preferred embodiment of the invention.

### DETAILED DESCRIPTION OF THE INVENTION

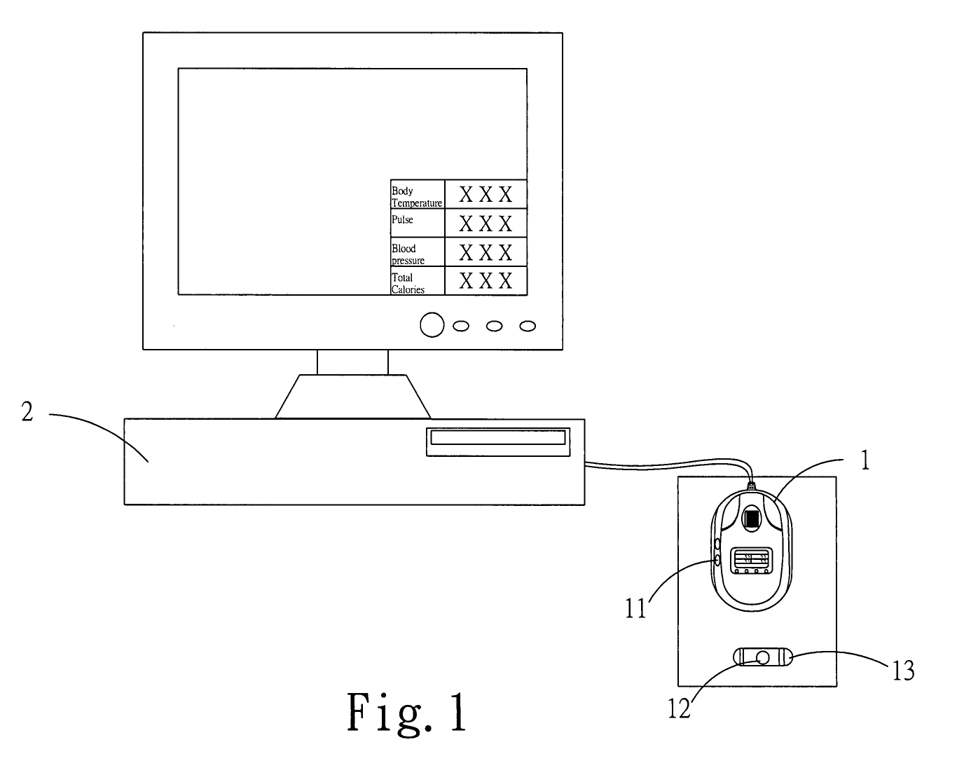

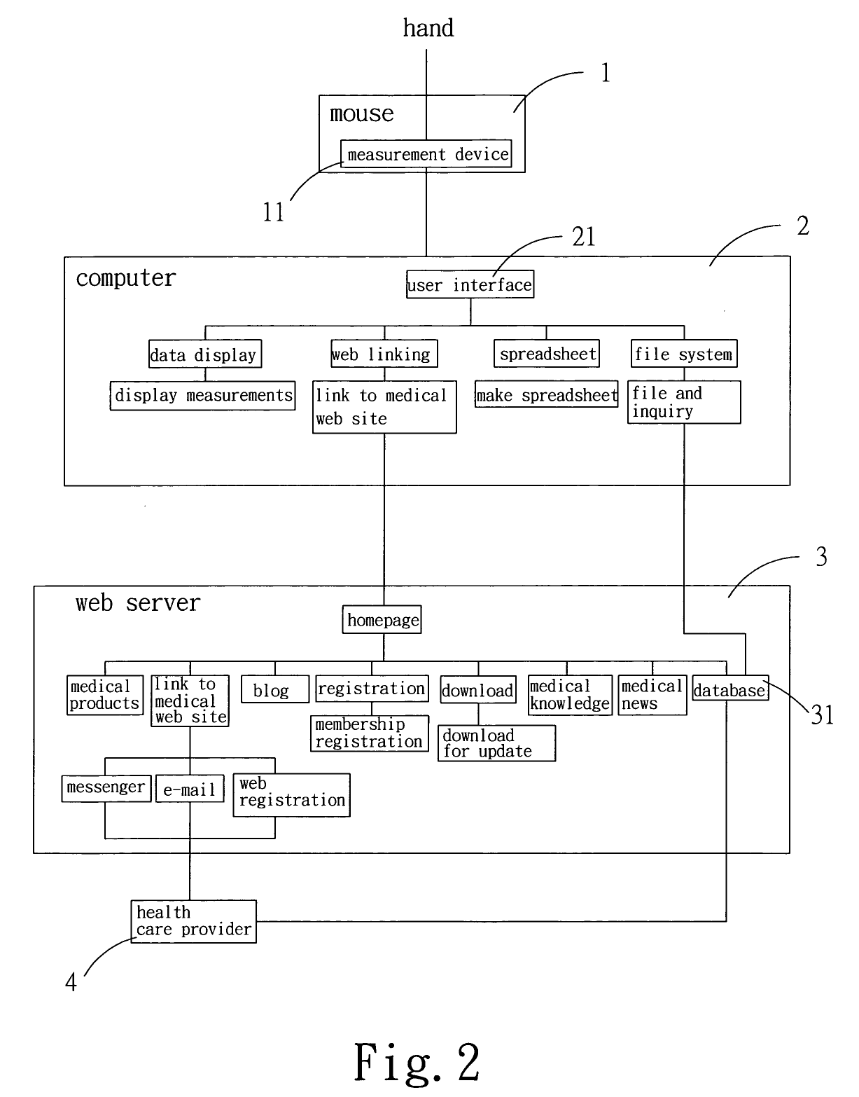

Referring to FIGS. 1 and 2, a system for measuring and displaying vital signs in accordance with a first preferred embodiment of the invention is shown. The system comprises a mouse 1 including a first measurement device 11 formed thereon, and an inflatable wrist band 13 including a second measurement device (e.g., sphygmomanometer) 12 formed therein. The wrist band 13 is mounted on a mouse pad (not numbered) with the mouse 1 placed thereon.

The system further comprises a health care provider (e.g., hospital) 4; a personal computer 2 including a display (not numbered) and an embedded user interface 21; and a web server 3 including a database 31 for user inquiry and linked to the health care provider 4 for providing data thereto. The web server 3 is further adapted to provide information about medical products, medical knowledge, and medical news, provide links to the health care provider 4 through e-mail or messenger, provide a web based reserved registration to the health care provider 4, provide a blog for discussion, provide membership registration, and download programs for update.

In operation, the wrist of a user is wrapped around by the wrist band 13 with the second measurement device 12 being in contact with the wrist. Hence, blood pressure of the user is measured by the second measurement device 12. Further, the hand holding the mouse 1 contacts the first measurement device 11. Hence, body temperature, pulse, and total calories of the user are measured by the first measurement device 11. Furthermore, data about the measurements is sent to the user interface 21 prior to sending to the display of the computer 2 for showing in the form of digits. Moreover, the user interface 21 is adapted to process data about the measured vital signs in a spreadsheet, process data into files to be stored in the database 31, and link to the health care provider 4 via the database 31.

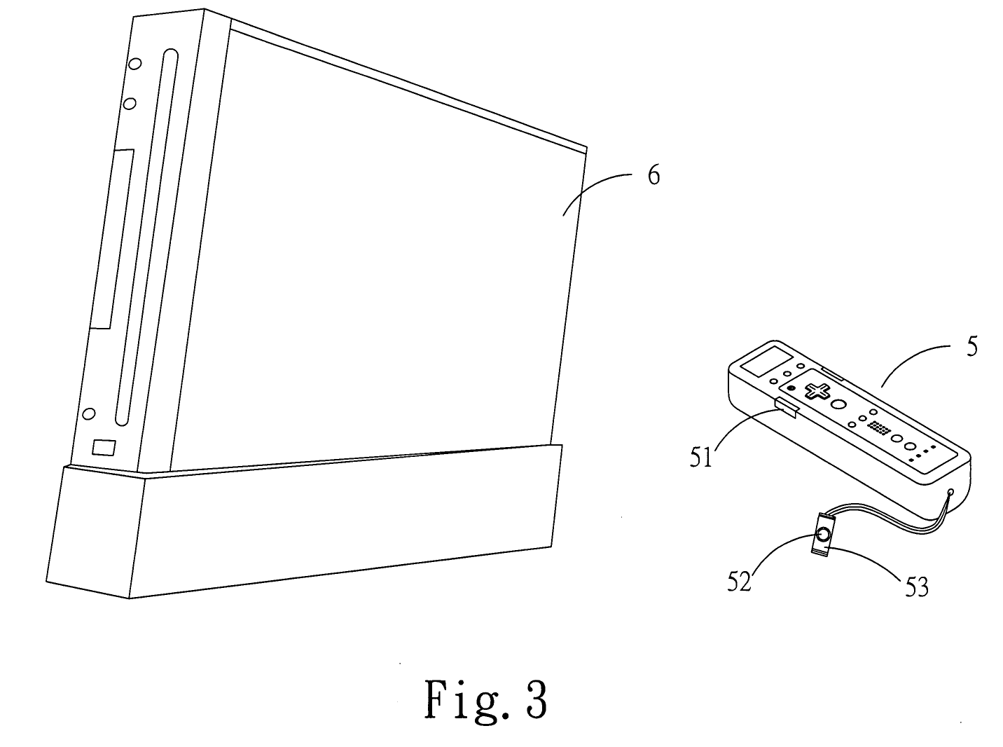

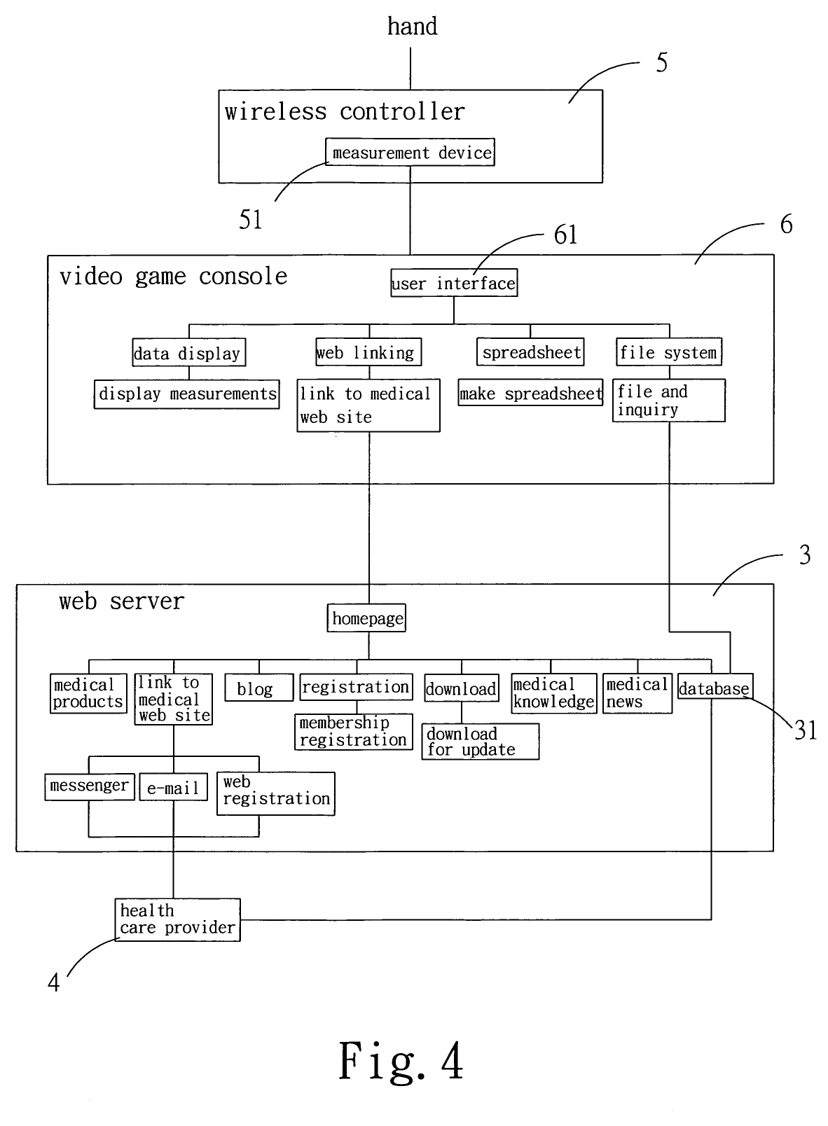

Referring to FIGS. 3 and 4, a system according to a second preferred embodiment of the invention is shown. The system comprises a video game console (e.g., “Wii”) 6, a wireless controller (e.g., “Wii” remote) 5 for controlling the video game console 6 and including a first measurement device 51 formed on one side edge, and an inflatable wrist band 53 including a second measurement device (e.g., sphygmomanometer) 52 formed therein. The second measurement device 52 is electrically connected to the circuitry of the wireless controller 5 via a cord.

The system further comprises a health care provider (e.g., hospital) 4, a monitor (not numbered), a user interface 61 embedded in the video game console 6, and a web server 3 including a database 31 for user inquiry and linked to the health care provider 4 for providing data thereto. The web server 3 is further adapted to provide information about medical products, medical knowledge, and medical news, provide links to the health care provider 4 through e-mail or messenger, provide a web based reserved registration to the health care provider 4, provide a blog for discussion, provide membership registration, and download programs for update.

In operation, the wrist of a user is wrapped around by the wrist band 53 with the second measurement device 52 being in contact with the wrist. Hence, blood pressure of the user is measured by the second measurement device 52. Further, the hand holding the wireless controller 5 contacts the first measurement device 51. Hence, body temperature, pulse, and total calories of the user are measured by the first measurement device 51 while the user is playing a video game by operating the wireless controller 5 with respect to the video game console 6. Furthermore, data about the measurements is sent to the user interface 61 prior to sending to the monitor for showing in the form of digits. Moreover, the user interface 61 is adapted to process data about the measured vital signs in a spreadsheet, process data into files to be stored in the database 31, and link to the health care provider 4 via the database 31.

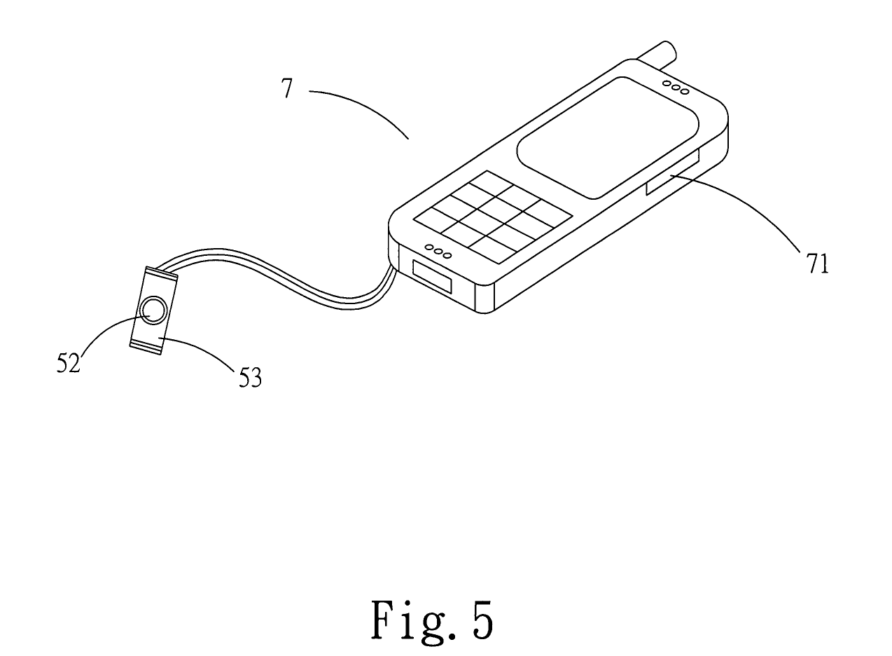

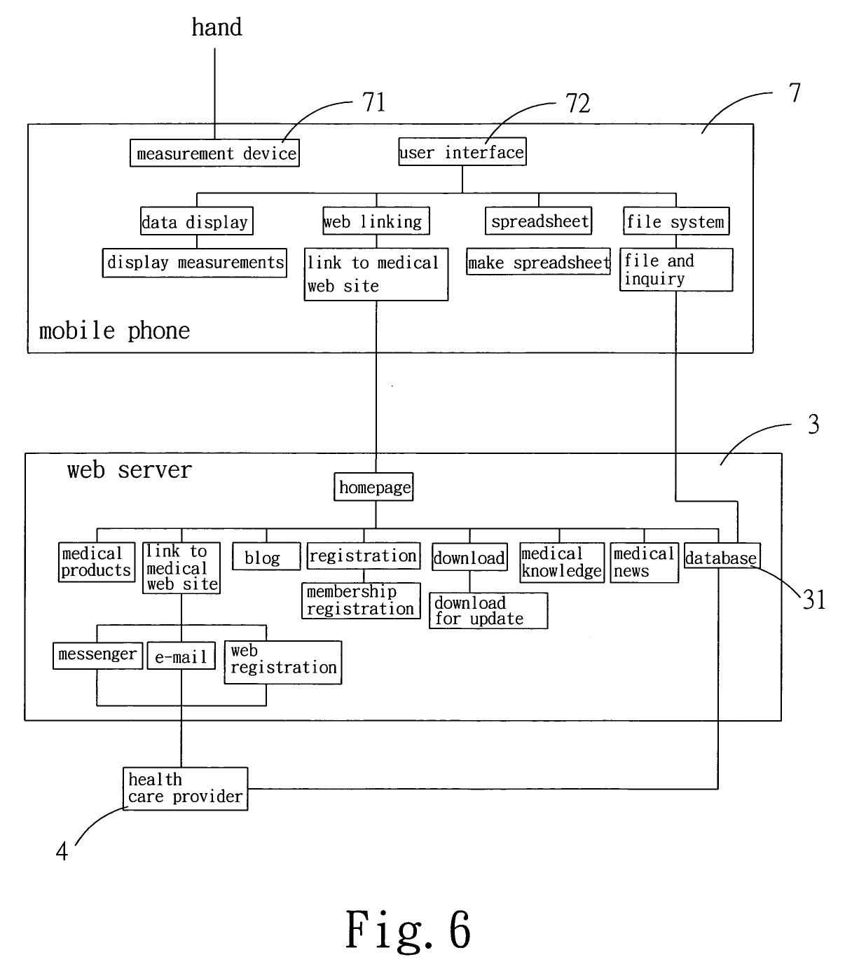

Referring to FIGS. 5 and 6, a system according to a third preferred embodiment of the invention is shown. The system comprises a mobile phone 7 including a first measurement device 71 formed on one side, and an inflatable wrist band 53 including a second measurement device (e.g., sphygmomanometer) 52 formed therein. The second measurement device 52 is electrically connected to the circuitry of the mobile phone 7 via a cord.

The system further comprises a health care provider (e.g., hospital) 4, a user interface 72 embedded in the mobile phone 7, and a web server 3 including a database 31 for user inquiry and linked to the health care provider 4 for providing data thereto. The web server 3 is further adapted to provide information about medical products, medical knowledge, and medical news, provide links to the health care provider 4 through e-mail or messenger, provide a web based reserved registration to the health care provider 4, provide a blog for discussion, provide membership registration, and download programs for update.

In operation, the wrist of a user is wrapped around by the wrist band 53 with the second measurement device 52 being in contact with the wrist. Hence, blood pressure of the user is measured by the second measurement device 52. Further, the hand holding the mobile phone 7 contacts the first measurement device 71. Hence, body temperature, pulse, and total calories of the user are measured by the first measurement device 71 while the user is making a telephone conversation by means of the mobile phone 7. Furthermore, data about the measurements is sent to the user interface 72 prior to sending to the display of the mobile phone 7 for showing in the form of digits. Moreover, the user interface 72 is adapted to process data about the measured vital signs in a spreadsheet, process data into files to be stored in the database 31, and link to the health care provider 4 via the database 31.

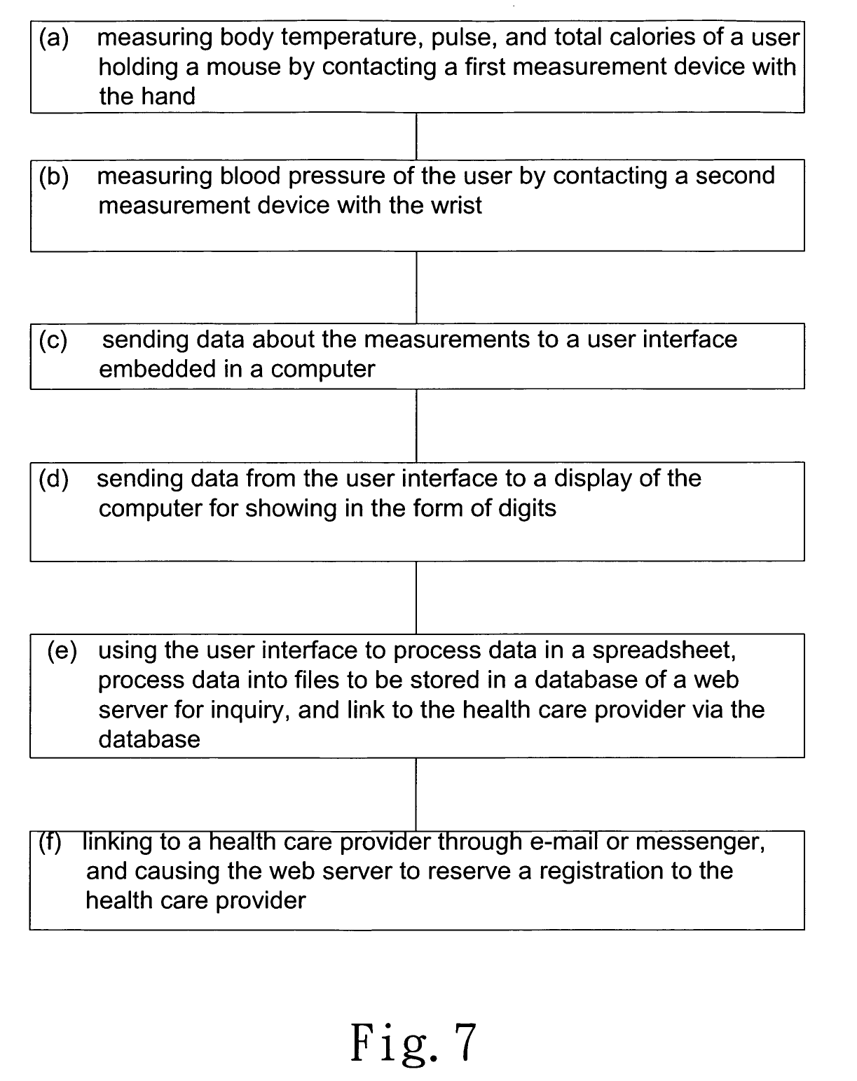

Referring to FIG. 7, a flowchart depicting a process for measuring and displaying vital signs in association with the system described in the first preferred embodiment of the invention is illustrated. The process comprises (a) measuring body temperature, pulse, and total calories of a user holding a mouse by contacting a first measurement device with the hand; (b) measuring blood pressure of the user by contacting a second measurement device with the wrist; (c) sending data about the measurements to a user interface embedded in a computer; (d) sending data from the user interface to a display of the computer for showing in the form of digital representation; (e) using the user interface to process data in a spreadsheet, process data into files to be stored in a database of a web server for inquiry, and link to the health care provider via the database; (f) linking to a health care provider through e-mail or messenger, and causing the web server to reserve a registration to the health care provider.

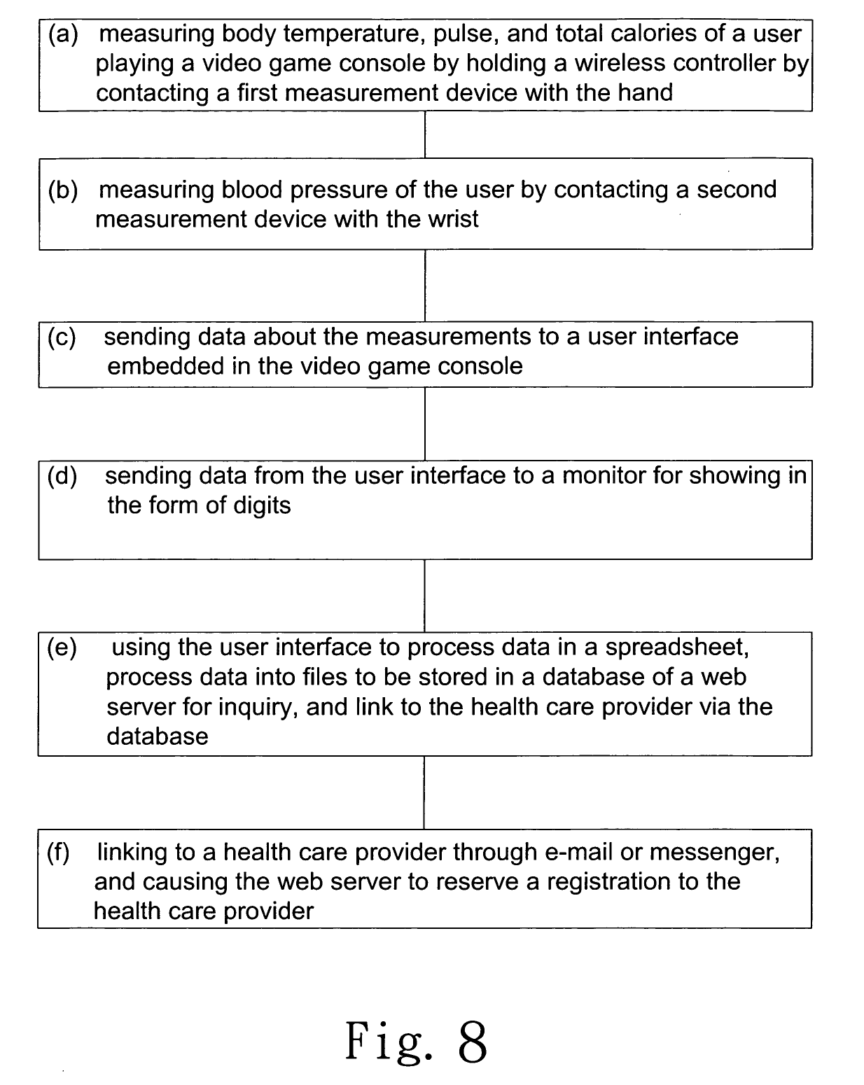

Referring to FIG. 8, a flowchart depicting a process for measuring and displaying vital signs in association with the system described in the second preferred embodiment of the invention is illustrated. The process comprises (a) measuring body temperature, pulse, and total calories of a user playing a video game console by holding a wireless controller by contacting a first measurement device with the hand; (b) measuring blood pressure of the user by contacting a second measurement device with the wrist; (c) sending data about the measurements to a user interface embedded in the video game console; (d) sending data from the user interface to a monitor for showing in the form of digital representation; (e) using the user interface to process data in a spreadsheet, process data into files to be stored in a database of a web server for inquiry, and link to the health care provider via the database; (f) linking to a health care provider through e-mail or messenger, and causing the web server to reserve a registration to the health care provider.

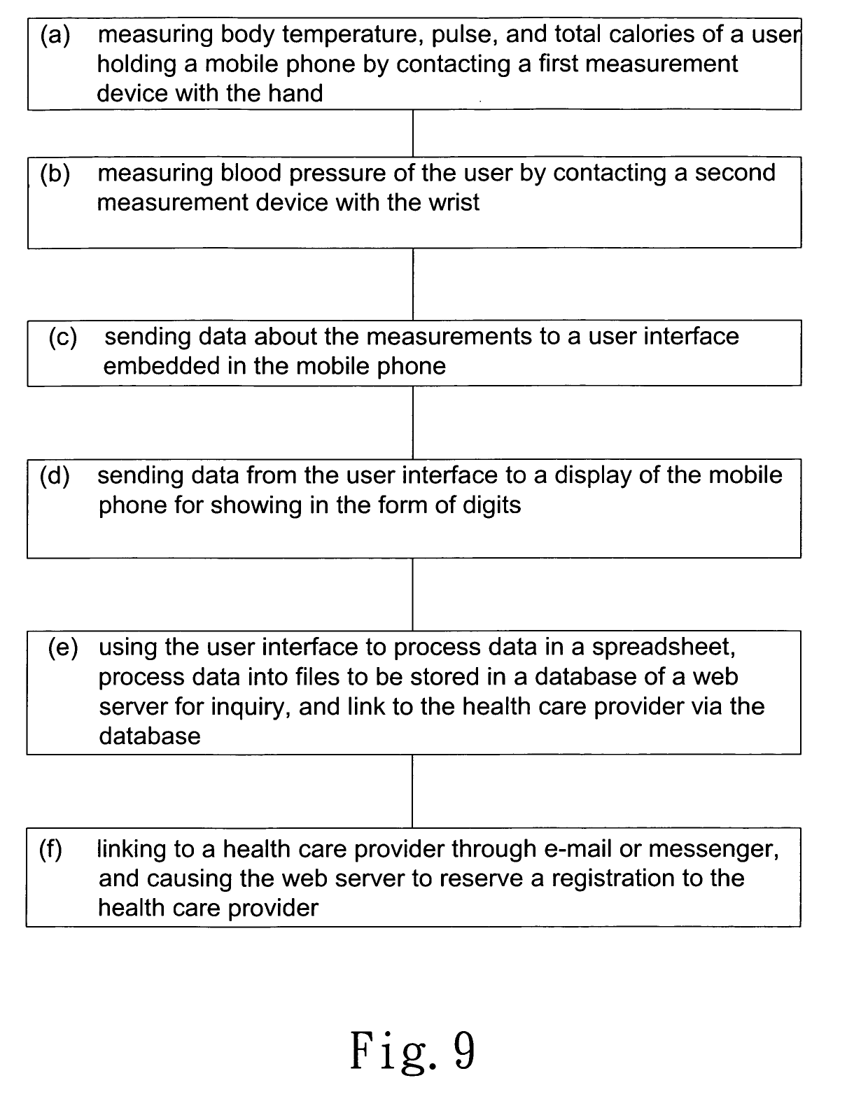

Referring to FIG. 9, a flowchart depicting a process for measuring and displaying vital signs in association with the system described in the third preferred embodiment of the invention is illustrated. The process comprises (a) measuring body temperature, pulse, and total calories of a user holding a mobile phone by contacting a first measurement device with the hand; (b) measuring blood pressure of the user by contacting a second measurement device with the wrist; (c) sending data about the measurements to a user interface embedded in the mobile phone; (d) sending data from the user interface to a display of the mobile phone for showing in the form of digital representation; (e) using the user interface to process data in a spreadsheet, process data into files to be stored in a database of a web server for inquiry, and link to the health care provider via the database; (f) linking to a health care provider through e-mail or messenger, and causing the web server to reserve a registration to the health care provider.

While the invention herein disclosed has been described by means of specific embodiments, numerous modifications and variations could be made thereto by those skilled in the art without departing from the scope and spirit of the invention set forth in the claims.

What is claimed is:

1. A system comprising:
a band wrapped around the wrist of a person and including a first measurement member being in contact with the wrist for measuring blood pressure of the person;
a device electrically connected to the band and including a second measurement member being in contact with the hand for measuring body temperature, pulse, and total calories of the person, a user interface, and a display;
a health care provider; and
a web server including a database;
wherein data about the measurements is sent to the user interface prior to sending to the display for showing in the form of digital representation.

2. The system of claim 1, wherein the user interface is adapted to process data about the measurements in a spreadsheet, process data about the measurements into files to be stored in the database, and link to the health care provider via the database.

3. The system of claim 1, wherein the device includes a mouse and a computer, and wherein the user interface is embedded in the computer and the display is a monitor of the computer.

4. The system of claim 1, wherein the device includes a video game console and a wireless controller, and wherein the user interface is embedded in the video game console and the display is a display of the wireless controller.

5. The system of claim 1, wherein the device includes a mobile phone, and wherein the user interface is embedded in the mobile phone and the display is a display of the mobile phone.

6. A method comprising:
measuring body temperature, pulse, and total calories of a person holding a device by contacting a first measurement member with the hand;
measuring blood pressure of the person by contacting a second measurement member with the wrist;
sending data about the measurements to a user interface embedded in the device;
sending data from the user interface to a display of the device for showing in the form of digital representation;
using the user interface to process data in a spreadsheet, process data into files to be stored in a database of a web server, and link to a health care provider via the database;
linking to the health care provider through e-mail or messenger; and
causing the web server to reserve a registration to the health care provider.

7. The method of claim 6, wherein the device includes a mouse and a computer, and wherein the user interface is embedded in the computer and the display is a monitor of the computer.

8. The method of claim 6, wherein the device includes a video game console and a wireless controller, and wherein the user interface is embedded in the video game console and the display is a display of the wireless controller.

9. The method of claim 6, wherein the device includes a mobile phone, and wherein the user interface is embedded in the mobile phone and the display is a display of the mobile phone.
